(19) United States
(12) Patent Application Publication
(10) Pub. No.: US 2014/0230822 A1
(43) Pub. Date: Aug. 21, 2014

(54) Forehead Abutting Device for Use with Respiratory Mask without Adjustment Thereof

(71) Applicant: Yen-Ting Kuo, Hsin Kang Towship (TW)

(72) Inventor: Yen-Ting Kuo, Hsin Kang Towship (TW)

(21) Appl. No.: 13/771,341

(22) Filed: Feb. 20, 2013

(51) Int. Cl. A61M 16/06

(52) U.S. Cl. CPC A61M 16/0683
USPC 128/206.21

(57) ABSTRACT

A forehead abutting device for use with a respiratory mask without adjustment thereof is provided. The upper end of the respiratory mask extends upward to form a support portion. A forehead cushion component, which has a forehead cushion and is capable of protracting and retracting resiliently and automatically, is disposed at the upper end of the support portion. The forehead cushion component includes a forehead cushion and a pipe portion extending therefrom. The terminal end of the pipe portion abuts against a receiving hole portion disposed at the upper end of the support portion. A resilient element capable of pushing the forehead cushion component outwardly and resiliently is disposed inside the receiving hole portion. The mask is fixed at three points and accurately positioned, fits different forehead contours through the forehead cushion component, imposes less burden on the nasal bridge, and prevents gas leakage.

### BACKGROUND OF THE INVENTION

1. Technical Field

The present invention relates to a forehead abutting device for use with a respiratory mask without adjustment thereof, wherein the respiratory mask is equipped with a forehead cushion component abutting against a user's forehead and being capable of protracting and retracting resiliently and automatically to thereby enable a forehead cushion to more easily adapt to the contour of the faces of different users and enable the respiratory mask to be worn by the users correctly.

2. Description of Related Art

The main cause of snoring is the obstruction of the upper respiratory tract, which can arise from obesity, senile regeneration of tissue, and the narrowing of the upper respiratory tract. While the patients with the aforesaid medical conditions are sleeping, the muscles in their throat relax; as a result, their upper respiratory tract collapses, thereby preventing air from entering the lungs and thus reducing the blood oxygen level. At this point in time, cerebral hypoxia triggers forceful breathing and thereby keeps the patients awake transiently. Hence, the patients are unable to sleep deeply and, as a result, the aforesaid symptom occurs to the patients dozens of times or even a hundred times each night; the medical condition is known as sleep apnea. Patients with sleep apnea alternate between a sleeping state and an awakened state repeatedly at night; as a result, the patients cannot enjoy high-quality sleep. Hence, the patients are likely to have headache as soon as they wake up in the morning. In the daytime, the patients manifest symptoms, such as sleepiness, drowsiness, inattention, memory loss, anxiety, sudden personality changes, and decreased libido. In the nighttime, the patients snore and urinate frequently and are likely to suffer cardiovascular disease attack.

Physicians usually recommend patients with mild sleep apnea do workouts, lose weight, practice side sleeping, refrain from alcohol and sleeping pills before going to bed, and wear a snore stopper (crown). Patients with severe sleep apnea have to be treated by surgery or a non-invasive positive pressure ventilator. The non-invasive positive pressure ventilator operates in conjunction with a mask and can cure more than 90% of the patients with severe sleep apnea. The patient wears the mask while sleeping, such that the positive pressure generated from the ventilator buttresses the otherwise flaccid upper respiratory tract to thereby keep the upper respiratory tract open. Nonetheless, as many as 30% to 40% of the patients quit non-invasive positive pressure ventilation treatment as they fail to endure the inconvenience brought by the mask and fail to tolerate wearing the mask for at least four straight hours a night, that is, the period of time required for the efficacy of non-invasive positive pressure ventilation treatment. The inconvenience brought by the mask include gas leakage, mask compression, exhalation resistance, weight of pipes, dry air flow, and being unportable.

Hence, it is not only important to make the patients more willing to receive non-invasive positive pressure ventilation treatment by solving the known problems with pipe weight, portability, and exhalation resistance, but also imperative to prevent a respiratory mask worn by a patient from angular displacement which might otherwise occur when the patient turns during sleep. To this end, two bands for fastening the mask in place can be tightened to thereby effectuate an airtight mask-to-face seal. To maintain the positive pressure inside the mask, a requisite of the mask has been that it provides an effective seal against the periphery of the patient's mouth and nose. However, if the mask-to-face seal is overly tight, it will cause discomfort to the patient.

In view of this, a structural design, whereby pressure is spread across the forehead and the periphery of the mouth and nose and thus shared by them, is put forth by U.S. Pat. No. 6,467,483 and U.S. Pat. No. 5,243,971 which disclose the following features: the upper end of the mask extends to form a support; a forehead cushion component capable of abutting against a user's forehead is disposed at the terminal end of the support; a head cord is fastened to each of the two sides of the forehead cushion component; the head cords can be tied around the user's head such that the mask is supported and fixed in place at three points, namely its flanks and top; and the support ensures that the mask is always perpendicular to the user's face and is unlikely to undergo angular displacement, so as to effectuate an airtight mask-to-face seal.

The relative positions of the mask, the forehead cushion component, and the support are constant rather than adjustable. As a result, in practice, the relative positions of the mask, the forehead cushion component, and the support cannot be adjusted to accommodate the many different forehead and facial contours likely to be encountered. Therefore, the seal between the forehead cushion component and a user's forehead is unsatisfactory, thereby resulting in gas leakage, overly low pressure, and discomfort.

### SUMMARY OF THE INVENTION

To overcome the aforesaid drawbacks of the prior art, that is, a conventional mask worn by a user has a support that abuts against the user's forehead, but the relative positions of the mask and the support are not adjustable and thus cannot suit different users, the present invention provides a forehead abutting device for use with a respiratory mask without adjustment thereof. A forehead cushion component capable of protracting and retracting resiliently and automatically is disposed at the upper end of a support portion of the mask, to enable the forehead cushion component to fit the foreheads of different users wearing the mask without adjustment thereof and enable the mask to be positioned well.

The technical solution of the present invention is described below. The upper end of a respiratory mask extends upward to form a support portion. A forehead cushion component with a forehead cushion is disposed at the upper end of the support portion. A receiving hole portion is disposed at the tip of the upper end of the support portion. An inner rim portion 12 whose aperture end is of a relatively small inner diameter is disposed at the receiving hole portion. The forehead cushion component comprises a forehead cushion, an engaging portion, and a pipe portion. The engaging portion is protrudingly disposed at the outer rim at the terminal end extending forward from the forehead cushion. The pipe portion extends forward from the front side of the forehead cushion and has a centrally-disposed receiving hole. The pipe portion extends into the receiving hole portion to allow the engaging portion to abut against the inner rim portion. A resilient element capable of pushing the forehead cushion component outwardly and resiliently is disposed inside the receiving hole portion.

The beneficial effects of the present invention are as follows: two head cords passing through the support portion from two sides thereof, coupled with the two bands fastened to the mask, give a three-point support to the mask and thereby fix the mask in place. The forehead cushion component capable of protracting and retracting resiliently and automatically enables the mask to fit flat foreheads and rounded foreheads of different users, ensures that the mask can be accurately positioned, lessens the burden imposed by the mask on the nasal bridge, and enhances the comfort of the mask.

### BRIEF DESCRIPTION OF THE DRAWINGS

The present invention is hereunder illustrated with an embodiment in conjunction with the accompanying drawings, in which:

FIG. 1 is a perspective view of a respiratory mask according to the present invention;

FIG. 2 is an exploded view of a forehead abutting device according to the present invention;

FIG. 3 is a cross-sectional view of the forehead abutting device according to the present invention; and

FIG. 4 is a schematic view of the compressed state of a forehead cushion component according to the present invention.

### DETAILED DESCRIPTION OF THE EMBODIMENT OF THE INVENTION

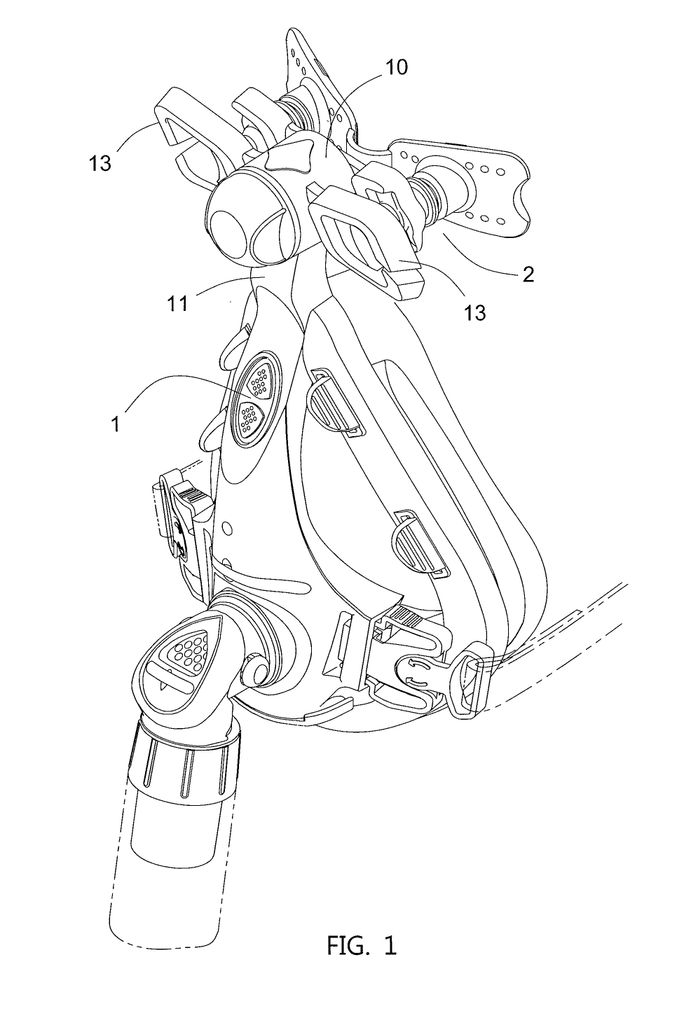

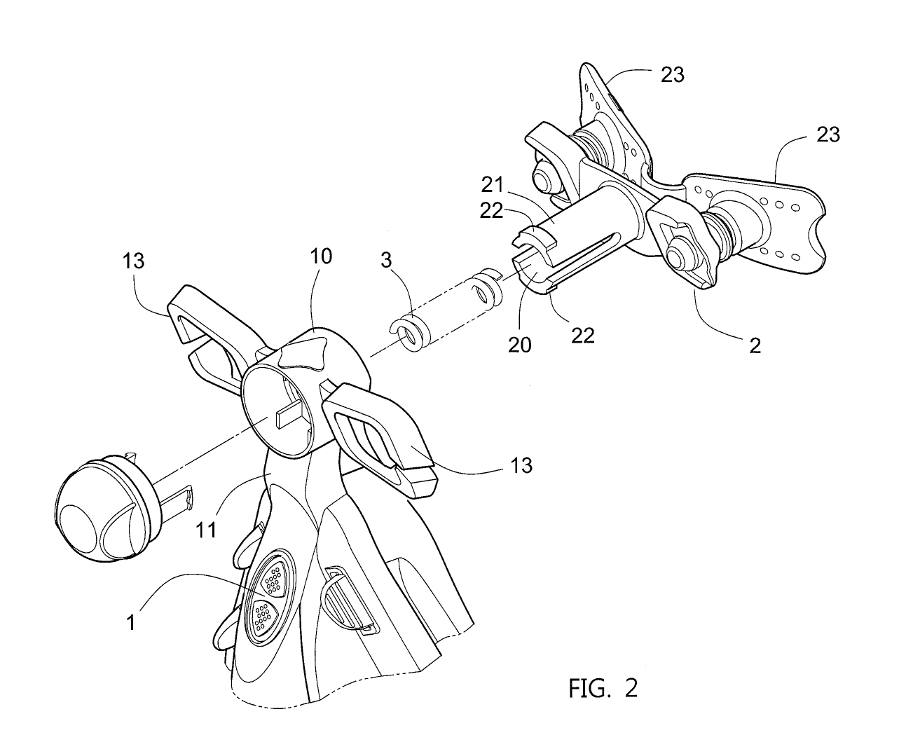

Referring to FIGS. 1-3, according to the present invention, a nasal portion at the upper end of a respiratory mask 1 extends upward to form a support portion 11. A receiving hole portion 10 is disposed at the terminal end of the support portion 11. Cord holders 13 through which a head cord can pass are disposed at two sides of the receiving hole portion 10, respectively. A forehead cushion component 2 for abutting against a user's forehead is disposed at the receiving hole portion 10. Hence, the support portion 11, the receiving hole portion 10, and the forehead cushion component 2 together form a forehead abutting device.

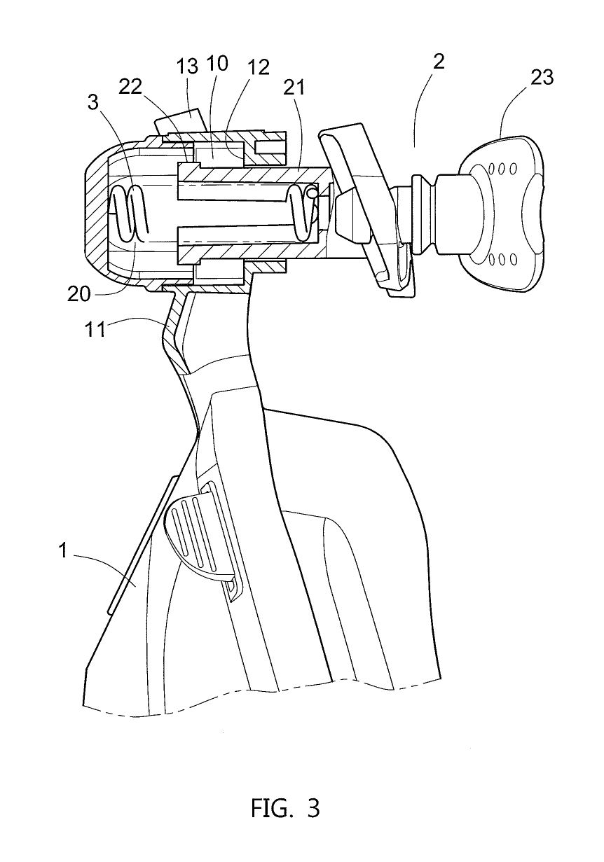

The forehead cushion component 2 comprises a forehead cushion 23 and a pipe portion 21. The pipe portion 21 extends forward from the front side of the forehead cushion 23 and has a centrally-disposed receiving hole 20. The pipe portion 21 is cut to thereby form a plurality of petal portions capable of opening and closing resiliently. An engaging portion 22 is protrudingly disposed at the outer rim at the terminal end of the pipe portion 21. An inner rim portion 12 (shown in FIG. 3) is disposed at the receiving hole portion 10 disposed at the upper end of the support portion 11. The inner rim portion 12 can be forcibly penetrated by the engaging portion 22 disposed at the terminal end of the pipe portion 21 and has a relatively small inner diameter. Afterward, a resilient element 3 capable of pushing the forehead cushion component 2 outwardly and resiliently is disposed between the pipe portion 21 and the receiving hole portion 10.

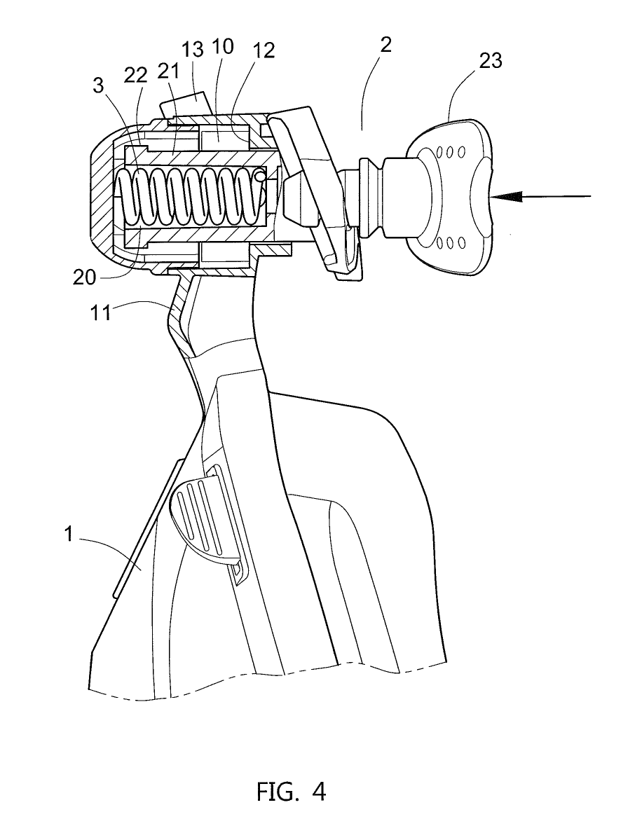

With the head cords passing through the cord holders 13 on two sides of the support portion 11, the upper end of the mask 1 is fixed in place. The head cords, coupled with the two bands fastened to the two sides of the mask 1, respectively, fix the mask 1 in place at three points, such that the mask 1 is positioned accurately and is unlikely to be displaced. Referring to FIG. 4, since the forehead cushion component 2 capable of protracting and retracting resiliently is disposed at the upper end of the support portion 11, the forehead cushion component 2 can be pushed up or pressed down by the resilient element 3 therein to thereby automatically protrude and abut against the flat forehead of a user or to thereby automatically retract and abut against the rounded forehead of another. Hence, not only is the mask 1 positioned accurately to thereby prevent gas leakage, but it is effective in lessening the burden imposed by the mask 1 on the nasal bridge, and dispensing with the need to adjust the forehead cushion component 2 in order to suit different users in terms of the accurate position of the mask 1 and the comfort thereof.

What is claimed is:

1. A forehead abutting device for use with a respiratory mask without adjustment thereof, the mask having an upper end extending to form a support portion, such that a forehead cushion component with a forehead cushion is disposed at a tip of an upper end of the support portion, the forehead abutting device being characterized in that: a receiving hole portion is disposed at the tip of the upper end of the support portion; an inner importion whose aperture end is of a relatively small inner diameter is disposed at the receiving hole portion; the forehead cushion component further comprises a forehead cushion, an engaging portion, and a pipe portion; the pipe portion extends forward from a front side of the forehead cushion, has a terminal end whose outer rim protruding to form the engaging portion, and has a centrally-disposed receiving hole; the pipe portion extends into the receiving hole portion to allow the engaging portion to abut against the inner rim portion and a resilient element capable of pushing the forehead cushion component outwardly and resiliently is disposed inside the receiving hole portion.
